SEQUENCE LISTING (5 sequences)

SEQ ID NO: 1 - DNA, 48 bp, artificial sequence
  shTyk2#1
cccatcttcattagctgggaactcgagttcccagctaatgaagatggg

SEQ ID NO: 2 - DNA, 48 bp, artificial sequence
  shTyk2#2
cccttcatcaagctaagtgatctcgagatcacttagcttgatgaaggg

SEQ ID NO: 3 - DNA, 48 bp, artificial sequence
  shTyk2#3
ccactttaagaatgagagcttctcgagaagctctcattcttaaagtgg

SEQ ID NO: 4 - DNA, 58 bp, artificial sequence
  Alk5#1 target sequence
ccggatagctgaaattgacctaattctcgagaattaggtcaatttcagctattttttg

SEQ ID NO: 5 - DNA, 58 bp, artificial sequence
  Alk5#2 target sequence
ccgggctgacagctttgcgaattaactcgagttaattcgcaaagctgtcagctttttg

(19) United States
(12) Patent Application Publication
(10) Pub. No.: US 2024/0384233 A1
(43) Pub. Date: Nov. 21, 2024

(54) Method for Producing Cardiomyocytes by Means of Reprogramming

(71) Applicants: Plastech Pharmaceutical Technology Co., Ltd., Jiangsu (CN); Peking University, Beijing (CN)

(72) Inventors: Yang Zhao, Beijing (CN); Yanmeng Tao, Beijing (CN); Jingdong Wu, Beijing (CN); Hao Wang, Beijing (CN)

(73) Assignees: Plastech Pharmaceutical Technology Co., Ltd., Jiangsu (CN); Peking University, Beijing (CN)

(21) Appl. No.: 18/018,569

(22) PCT Filed: Jul. 29, 2021
(86) PCT No.: PCT/CN2021/109183

§ 371 Date: Mar. 1, 2024

(30) Foreign Application Priority Data
Jul. 29, 2020 (WO) PCT/CN2020/105421

(51) Int. Cl. C12N 5/077; C12N 15/86

(52) U.S. Cl. CPC C12N 5/0657; C12N 15/86; C12N 2501/60; C12N 2501/999; C12N 2506/1307; C12N 2740/15043

(57) ABSTRACT

The present invention relates to the field of biomedicine, in particular regenerative medicine. In particular, the present invention relates to a method for producing cardiomyocytes from differentiated cells, such as fibroblasts, by means of reprogramming using a Tyk2 inhibitor and/or a TGFβ inhibitor, and optionally a cardiomyocyte-inducing transcription factor.

### TECHNICAL FIELD

The present invention relates to the field of biomedicine, in particular regenerative medicine. In particular, the present invention relates to a method for producing cardiomyocytes from differentiated cells, such as fibroblasts, by means of reprogramming using a Tyk2 inhibitor and/or a TGFβ inhibitor, and optionally a cardiomyocyte-inducing transcription factor.

### BACKGROUND ART

23 million people worldwide suffer from heart failure, which is usually caused by myocardial cell injury or dysfunction. One common cause of cardiomyocyte loss is ischemic heart disease leading to myocardial infarction, which is permanent and progressive due to limited cardiac regenerative capacity. Despite advances in medical treatment, there is currently no strategy for restoring muscle mass other than orthotopic heart transplantation, which is limited by the number of cell sources and long-term efficacy. To date, cell therapy used in human trials has demonstrated that transplanted cells do not become cardiomyocytes in large numbers, and do not persist in the heart. Cardiomyocyte transplantation from pluripotent stem cells is being tested and may be valuable if problems such as cell survival, maturation and electrophysiological integration are effectively addressed. In situ reprogramming of cells to cell types lost in disease using cell type-specific transcription factors (TFs) is a promising alternative to cell therapy for effective tissue regeneration.In the heart, a large number of non-cardiomyocytes, mainly cardiac fibroblasts, can be transformed into induced cardiomyocyte-like cells by transcription factors.

In rodents, three cardiac-specific TFs: GATA4, MEF2C and TBX5 (GMT) were injected directly into myocardium after coronary ligation, resulting in the transformation of non-cardiomyocytes into cardiomyocyte-like cells that are electrically coupled to existing cardiomyocytes, thereby improving cardiac function and reducing scar size. However, efficiency is still limited, especially in vitro, where most cells are not completely reprogrammed The signal existing in vivo that leads to the improvement of reprogramming quality is not clear, but suggests that altering culture conditions or signaling pathways may enhance cardiac reprogramming in vitro (and possibly in vivo).

Since the success of first generation cardiomyocyte reprogramming, there have been many reports on enhancing the efficiency of cardiac reprogramming By altering the metrology of the three genes of GMT, identifying additional genes on the basis of GMT, or by manipulating signaling pathways, both the quality and efficiency of cardiomyocyte-like cells generated in vitro can be improved. In most cases, the increase in efficiency is mainly found in mouse embryonic fibroblasts; in contrast, reprogramming initiated on cardiac fibroblasts and adult skin is limited. Although the recent siRNA-mediated knock-out of the bmi1 gene, and the strategy of combining SB431542 with XAV939, have improved the efficiency of reprogramming cardiac fibroblasts in vitro, it remains to be determined whether methods exist to further enhance reprogramming in mice or affect reprogramming of human cardiac fibroblasts.

Therefore, there remains a need in the art for new reprogramming methods that can efficiently generate functional cardiomyocytes by reprogramming in vivo or in vitro to treat cardiac diseases such as heart failure.

### BRIEF DESCRIPTION OF THE DRAWINGS

FIG. 1. Small molecules that promote cardiomyocyte reprogramming were identified. A. Strategies for screening small molecules that promote cardiomyocyte reprogramming; B. Myh6-mCherry-positive cell fluorescence image.C. Myh6-mCherry positive cell counts.The results show that two small molecules SB431542 and Baricitinib (2C) synergistically promoted the transdifferentiation of fibroblasts to induced cardiomyocyte like cells (iCMs).

FIG. 2 shows the optimal action concentration and optimal action time of 2C.

FIG. 3 shows a comparison of 2C with SB431542+XAV939 in efficiency of promoting iCM.

FIG. 4 shows that GMT+2C significantly improved reprogramming efficiency and quality.

FIG. 5 shows that MT+2C could induce cells with mature morphology, expression of typical cardiomyocyte-specific genes, spontaneous calcium transients, and action potentials similar to ventricular-type cardiomyocytes.

FIG. 6 shows that Gata4 could be effectively replaced only under the joint action of 2C.

FIG. 7 shows RNA-seq data, indicating that 2C could significantly up-regulate cardiac muscle-related genes while down-regulating fibroblast-related genes on the basis of GMT or MT.

FIG. 8 shows the principal component analysis of 782 genes with the largest difference in RNA-seq results.Addition of 2C allowed the GMT or MT-induced cardiomyocytes to attain a global cellular state closer to that of adult cardiomyocytes.

FIG. 9 shows GO analysis for genes that were significantly up-regulated and down-regulated by GMT+2C vs GMT.

FIG. 10 shows that SB431542 could be replaced by small molecules of the same signaling pathway.

FIG. 11 shows the failure of Dexamethasone or Nabumetone to replace Baricitinib.

FIG. 12 shows that Baritinib structural analogue had the effect of promoting cardiomyocyte reprogramming

FIG. 13 shows that 2C significantly improved the efficiency of reprogramming human fibroblasts into cardiomyocytes on the basis of 5 transcription factors.

FIG. 14 shows that 2C could subtract one transcription factor from the 5 transcription factors reported to induce hiCM.

FIG. 15 shows the schematic diagram of reprogramming in vivo, using Postn to trace cardiac myofibroblasts after myocardial infarction.

FIG. 16 shows that 2C significantly improved the efficiency of in situ reprogramming in vivo, and significantly improved the myocardial infarction edge area and infarction area of the mouse myocardial infarction model.

FIG. 17 shows the results of Masson trichrome staining of mouse myocardial infarction model, indicating that 2C significantly reduced the area of cardiac fibrosis (red is muscle fiber, blue is collagen).

FIG. 18 shows that in the mouse myocardial infarction model, compared with EGFP control group, only 2C treatment had significant reprogramming phenomenon, and reached the same reprogramming efficiency only in MGT group.

FIG. 19 shows the results of Masson trichrome staining in the mouse myocardial infarction model, showing that only 2C treatment could significantly reduce the area of fibrosis.

FIG. 20 shows that the combination of Ruxolitinib and SB43152 had better myocardial reprogramming effect in vivo.

FIG. 21 shows a schematic diagram of testing the effect of knocking down Tyk2 on cardiomyocyte reprogramming A: experimental design diagram, with neonatal mouse fibroblasts as starting cells, which were reprogrammed into cardiomyocytes only in the presence of reprogramming medium (C1 added) under the condition of infection with transcription factor MT combination and shRNA. B: specific experimental steps of reprogramming C: knockdown effect of Tyk2.

FIG. 22 shows that under the condition of MT+SB, a large number of cardiac specific markers cTnI and a-actinin positive cells were induced from fibroblasts by knocking down Tyk2.

FIG. 23 shows the expression level of cardiac specific markers of reprogrammed cells detected by qPCR.

FIG. 24 shows that knockout of Tyk2 by CRISPR promoted cardiomyocyte reprogramming induced by transcription factor MT and small molecule compound C1.

FIG. 25 shows that Tyk2 small molecule inhibitor BMS-986165 and/or PF-06826647 could induce a number of cardiac specific markers cTnI and a-actinin positive cells from fibroblasts.

FIG. 26 shows that Tyk2 small molecule inhibitor BMS-986165 and/or PF-06826647 could induce beating cardiomyocytes from fibroblasts.

FIG. 27 shows that Tyk2 inhibitor Ruxolitinib and TGFβ inhibitor TEW-7197 improved cardiac in situ reprogramming efficiency.

FIG. 28 shows that Tyk2 inhibitor Ruxolitinib and TGFβ inhibitor TEW-7197 improved cardiac fibrosis after MI.

FIG. 29 shows that SB431542 and Baricitinib (2C) in combination with MYOCD resulted in improved hiCM induction efficiency.

FIG. 30 shows that under the condition of MT+SB, a large number of cardiac specific markers cTnI and a-actinin positive cells were induced from fibroblasts by knocking down TGFβ receptor Alk5.

FIG. 31 shows that 2C significantly improved cardiac function in vivo.

### DETAILED DESCRIPTION OF THE INVENTION

The inventors performed a small molecule screening on mouse cardiac fibroblasts and revealed a novel method that can enhance cardiomyocyte reprogramming This method can significantly improve the efficiency of direct reprogramming of cardiomyocytes mediated by the transcription factor combination GMT in vitro and in vivo through the combination of a Tyk2 inhibitor and/or a TGFβ inhibitor. The present application demonstrates that GMT-induced cardiomyocyte reprogramming can be 100-fold more efficient by combining these two types of small molecules. The combination of small molecules can accelerate the reprogramming process and improve the quality of the obtained cardiomyocyte-like cells, in particular shorten beating time of cardiomyocytes and increase the proportion of beating cells. The combination of small molecules can also reduce the number of exogenous transcription factors required for reprogramming without reducing the efficiency and quality of reprogramming. Experiments on human cells have also demonstrated that this combination of small molecules can increase transcription factor-mediated reprogramming efficiency by 20-fold and can reduce the number of transcription factors required for reprogramming from 5 to 4. These findings demonstrate the great potential of gene therapy and drug combination therapy for cardiac regeneration in vivo.

In one aspect, the present invention provides a method for reprogramming a starting cell into a cardiomyocyte, the method comprising contacting the starting cell with at least one Tyk2 inhibitor and/or at least one TGFβ inhibitor.

As used herein, an “Tyk2” inhibitor refers to a substance that inhibits the Tyk2 signaling pathway, such as inhibitory antibodies, small molecule compounds, and the like, including, but not limited to Baricitinib, Ruxolitinib, S-Ruxolitinib, Tofacitinib, Oclacitinib maleate, Itacitinib, Peficitinib, Gandotinib, FM-381, Filgotinib, PF-06826647, BM S-986165, or structural analogs thereof. In some embodiments, the Tyk2 inhibitor is Baricitinib. In some embodiments, the Tyk2 inhibitor is Ruxolitinib. In some embodiments, the Tyk2 inhibitor is PF-06826647. In some embodiments, the Tyk2 inhibitor is BMS-986165.

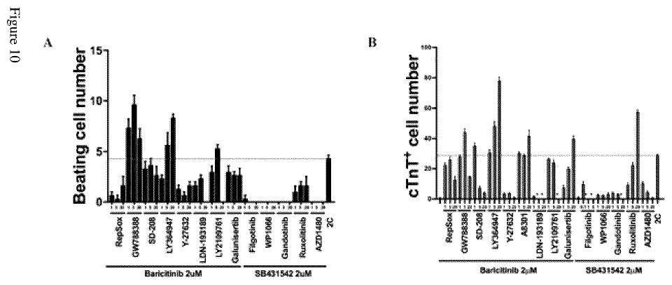

It is noted that all references herein to small molecule compounds encompass pharmaceutically acceptable salts thereof. For example, Ruxolitinib includes Ruxolitinib phosphate, while Tofacitinib also covers Tofacitinib citrate. The chemical structures of some of the Tyk2 inhibitors exemplified herein can be found in FIG. 12.

As used herein, a “TGFβ inhibitor” refers to a substance that inhibits the TGF β signaling pathway, such as inhibitory antibodies, small molecule compounds, and the like, including, but not limited to SB43152, TEW-7197, RepSox, GW788388, SD-208, LY364947, Y-27632, LDN-193189, LY2109761, and Galunisertib, or structural analogs thereof. In some embodiments, the TGFβ inhibitor is SB43152. In some embodiments, the TGFβ inhibitor is TEW-7197.

In some embodiments, the method comprises contacting the starting cell with a Tyk2 inhibitor and a TGFβ inhibitor.

In some embodiments, the at least one Tyk2 inhibitor comprises 1, 2, 3 or more Tyk2 inhibitors. In some embodiments, the at least one TGFβ inhibitor comprises 1, 2, 3 or more Tyk2 inhibitors.

In some embodiments, the method comprises contacting the starting cell with Baricitinib and SB43152.

In some embodiments, the method comprises contacting the starting cell with Ruxolitinib and TEW-7197. In some embodiments, the method comprises contacting the starting cell with Ruxolitinib and SB43152. In some embodiments, the method comprises contacting the starting cell with PF-06826647 and SB43152. In some embodiments, the method comprises contacting the starting cell with BMS-986165 and SB43152. In some embodiments, the method comprises contacting the starting cell with PF-06826647, BMS-986165, and SB43152.

In some embodiments, the starting cell is a differentiated cell. In some embodiments, the starting cell is a non-cardiomyocyte. The starting cell may be a mesodermal-derived cell such as a cardiac cell, an ectodermal-derived cell such as a nerve cell, or an endodermal-derived cell such as a colon cell. In some embodiments, the starting cell is a neuronal cell, a skeletal muscle cell, a hepatocyte, a fibroblast, an osteoblast, a chondrocyte, an adipocyte, an endothelial cell, a mesenchymal cell, a smooth muscle cell, a cardiomyocyte, a neural cell, a hematopoietic cell, an islet cell, or virtually any cell in the body. In some embodiments, the starting cell is a skin fibroblast. In some embodiments, the starting cell is a cardiac fibroblast.

In some embodiments, the starting cell is an isolated cell (ex vivo cell).

In the present invention, the starting cell may be derived from a mammal or a non-mammal. In some embodiments of the present invention, the starting cell is derived from human. In some embodiments of the present invention, the starting cell is derived from a non-human mammal. In some embodiments of the present invention, the starting cell is derived from a murine such as a mouse or a rat or non-human primate.

In some embodiments, the reprogrammed cardiomyocyte is a functional cardiomyocyte. The functional cardiomyocyte has, for example, one or more of the following characteristics: α-actinin positive, cTnT positive, with an ordered sarcomere structure, beating, expression of ventricular-type cardiomyocyte markers such as Myl2v, spontaneous calcium transient, action potential similar to ventricular-type cardiomyocyte, etc.

In the present invention, “contacting the starting cell with a Tyk2 inhibitor and/or a TGFβ inhibitor” may be achieved, for example, by culturing the starting cell in a medium comprising the Tyk2 inhibitor and/or the TGFβ inhibitor.

In some embodiments, the concentration of the Tyk2 inhibitor such as Baricitinib is from about 0.1 μM to about 50 μM, e.g. about 0.1 μM, about 0.5 μM, about 1 μM, about 1.5 μM, about 2 μM, about 2.5 μM, about 5 μM, about 7.5 μM, about 10 μM, about 15 μM, about 20 μM, about 30 μM, about 40 μM, about 50 μM. In some preferred embodiments, the concentration of the Tyk2 inhibitor such as Baricitinib is about 2 μM. In some preferred embodiments, the concentration of the Tyk2 inhibitor such as PF-06826647 or BMS-986165 is about 5 μM.

In some embodiments, the concentration of the TGFβ inhibitor such as SB43152 is from about 0.1 μM to about 50μM, e.g. about 0.1 μM, about 0.5μM, about 1μM, about 1.5 μM, about 2 μM, about 2.5μM, about 5μM, about 7.5μM, about 10 μM, about 15μM, about 20 μM, about 30 μM, about 40μM, about 50 μM. Preferably, the concentration of the TGFβ inhibitor such as SB43152 is about 2 μM.

In some embodiments, the method of the present invention comprises “contacting the starting cell with a Tyk2 inhibitor and/or a TGFβ inhibitor” for about 1 day to about 21 days or more, e.g. for about 3 days to about 21 days or more, for about 6 days to about 21 days or more, for about 9 days to about 21 days or more, for about 12 days to about 21 days or more, for about 15 days to about 21 days or more, or for about 18 days to about 21 days or more.

In some embodiments, the method further comprises providing at least one cardiomyocyte-inducing transcription factor and/or at least one cardiomyocyte-inducing microRNA to the starting cell.

As used herein, “cardiomyocyte-inducing transcription factor” refers to a transcription factor that, upon introduction into a starting cell, is capable of causing the starting cell to reprogram into a cardiomyocyte under appropriate conditions. A variety of transcription factors are known in the art that can be used to generate cardiomyocytes by reprogramming, including but not limited to: MEF2C, TBX5, GATA4, MESP1, MYOCD, HAND2, SRF, ESRRG, ZFPM2, Nkx2.5, VEGF, Baf60c, and any combination thereof.

In some embodiments, the at least one cardiomyocyte-inducing transcription factor comprises at least MEF2C.

In some embodiments, the at least one cardiomyocyte-inducing transcription factor further comprises TBX5. For example, the at least one cardiomyocyte-inducing transcription factor comprises, or consists of, MEF2C and TBX5.

In some embodiments, the at least one cardiomyocyte-inducing transcription factor further comprises GATA4. For example, the at least one cardiomyocyte-inducing transcription factor comprises, or consists of, MEF2C, TBX5, and GATA4.

In some embodiments, the at least one cardiomyocyte-inducing transcription factor further comprises MYOCD. For example, the at least one cardiomyocyte-inducing transcription factor comprises, or consists of, MEF2C, TBX5, GATA4, and MYOCD.

In some embodiments, the at least one cardiomyocyte-inducing transcription factor further comprises MESP1. For example, the at least one cardiomyocyte-inducing transcription factor comprises, or consists of, MEF2C, TBX5, GATA4, MYOCD, and MESP1.

In some embodiments, the at least one cardiomyocyte-inducing transcription factor comprises, or consists of, MEF2C, GATA4, MYOCD, and MESP1.

In some embodiments, the at least one cardiomyocyte-inducing transcription factor is MYOCD.

As used herein, “cardiomyocyte-inducing microRNA” refers to a microRNA that, upon introduction into a starting cell, is capable of causing the starting cell to reprogram into a cardiomyocyte under appropriate conditions. A variety of microRNA are known in the art that can be used to generate cardiomyocytes by reprogramming, including but not limited to: miR1, miR133, miR208, and miR499, and any combination thereof. In some embodiments, the at least one cardiomyocyte-inducing microRNA comprises, or consists of, miR1, miR133. In some embodiments, the at least one cardiomyocyte-inducing microRNA comprises or consists of miR1, miR133, miR208, and miR499.

The at least one cardiomyocyte-inducing transcription factor and/or the at least one cardiomyocyte-inducing microRNA may be provided to the starting cell, i.e. introduced into the starting cell, by any method known in the art.

For example, an expression vector comprising a nucleotide sequence encoding the transcription factor and/or microRNA may be introduced into the starting cell. Methods for introducing expression vectors into cells are known in the art and include, but are not limited to, DEAE-dextran method, calcium phosphate method, cationic liposome method, cationic polymer, Biolistic particle delivery method (gene gun particle bombardment), microinjection method, electroporation method, and virus-mediated method. Wherein preferably the expression vector is a viral expression vector, introduction of a nucleotide sequence encoding the transcription factor and/or microRNA may be effected by viral transfection. The viral vector is preferably a lentiviral vector, a retroviral vector, an adenoviral vector or the like. Methods for constructing viral vectors, such as lentiviral vectors, comprising a desired nucleotide sequence are known in the art.

In some embodiments of the method of the present invention, the step of “providing the starting cell with at least one cardiomyocyte-inducing transcription factor and/or at least one cardiomyocyte-inducing microRNA” may be performed before or after or simultaneously with the step of “contacting the starting cell with a Tyk2 inhibitor and/or a TGFβ inhibitor”, preferably before. For example, the step of “providing the starting cell with at least one cardiomyocyte-inducing transcription factor and/or at least one cardiomyocyte-inducing microRNA” may be performed one day before the step of “contacting the starting cell with at least one Tyk2 inhibitor and/or at least one TGFβ inhibitor”.

In one aspect, the present invention provides use of a Tyk2 inhibitor and/or a TGFβ inhibitor as hereinbefore described in the preparation of a reagent or kit for preparing a cardiomyocyte from a starting cell. The Tyk2 inhibitor and the TGFβ inhibitor are as defined above.

In one aspect, the present invention provides a cardiomyocyte prepared by the method of the present invention.

In one aspect, the present invention provides a pharmaceutical composition comprising a cardiomyocyte prepared by the method of the present invention and a pharmaceutically acceptable carrier.

In one aspect, the present invention also provides use of a cardiomyocyte prepared by the method of the present invention or a pharmaceutical composition of the invention comprising a cardiomyocyte prepared by the method of the present invention and a pharmaceutically acceptable carrier in the preparation of a medicament for the treatment of a cardiac disease. The cardiac disease is particularly myocardial disease, including but not limited to heart failure, myocardial infarction and the like.

In one aspect, the present invention further provides a method for treating a cardiac disease in a subject, the method comprising administering to the subject a cardiomyocyte prepared by the method of the present invention or a pharmaceutical composition of the invention comprising a cardiomyocyte prepared by the method of the present invention and a pharmaceutically acceptable carrier.

As used herein, a “subject” may be a mammal or a non-mammal The subject may be a human, or a non-human mammal such as a mouse or a rat or a non-human primate.

Furthermore, the inventors have surprisingly found that in vivo treatment of post-myocardial infarction mice with GMT, a TGFβ inhibitor such as SB43152, and a Tyk2 inhibitor such as Baricitinib could improve in vivo reprogramming efficiency in situ and effectively reduce scar area. More surprisingly, after treatment of myocardial infarction mice with a small molecule TGFβ inhibitor such as SB431542 and a Tyk2 inhibitor such as Baricitinib alone, in situ reprogramming was also be observed, and the in vivo reprogramming efficiencies were comparable to that with transcription factor (GMT) alone.

Accordingly, in one aspect, the present invention also provides a method for treating a cardiac disease in a subject, the method comprising administering to the subject at least one Tyk2 inhibitor and/or at least one TGFβ inhibitor. The cardiac disease is particularly myocardial disease, including but not limited to heart failure, myocardial infarction and the like. The Tyk2 inhibitor and the TGFβ inhibitor are as defined above.

In some embodiments, the method further comprises administering to the subject at least one cardiomyocyte-inducing transcription factor and/or at least one cardiomyocyte-inducing microRNA. The “at least one cardiomyocyte-inducing transcription factor” and “at least one cardiomyocyte-inducing microRNA” are as defined above. In some embodiments, the “administering at least one cardiomyocyte-inducing transcription factor and/or at least one cardiomyocyte-inducing microRNA” comprises administering an expression vector, e.g. a viral vector, preferably a lentiviral vector, comprising a nucleotide sequence encoding the transcription factor and/or microRNA.

In some embodiments, the administration is systemic. In some embodiments, the administration is topical, e.g. intracardiac.

In one aspect, the present invention provides a pharmaceutical composition comprising at least one Tyk2 inhibitor and/or at least one TGFβ inhibitor as defined herein and a pharmaceutically acceptable carrier. In some embodiments, the pharmaceutical composition further comprises an expression vector, e.g. a viral vector, preferably a lentiviral vector, comprising at least one cardiomyocyte-inducing transcription factor and/or at least one cardiomyocyte-inducing microRNA as defined herein, or comprising a nucleotide sequence encoding the transcription factor and/or microRNA.

In one aspect, the present invention provides use of at least one Tyk2 inhibitor and/or at least one TGFβ inhibitor as defined in the present invention for the preparation of a medicament for the treatment of a cardiac disease. The cardiac disease is particularly myocardial disease, including but not limited to heart failure, myocardial infarction and the like.

In one aspect, the present invention provides use of at least one Tyk2 inhibitor and/or at least one TGFβ inhibitor as defined herein, and at least one cardiomyocyte-inducing transcription factor and/or at least one cardiomyocyte-inducing microRNA as defined herein, or an expression vector comprising a nucleotide sequence encoding the transcription factor and/or microRNA, for the preparation of a medicament for the treatment of a cardiac disease. The expression vector is e.g. a viral vector, preferably a lentiviral vector. The cardiac disease is particularly myocardial disease, including but not limited to heart failure, myocardial infarction and the like.

In one aspect, the present invention provides a reprogramming medium comprising at least one Tyk2 inhibitor and/or at least one TGFβ inhibitor as defined herein. In some embodiments, the reprogramming medium is used in the method of the present invention.

In one aspect, the present invention provides a kit for reprogramming a starting cell into a cardiomyocyte, the kit comprising at least one Tyk2 inhibitor and/or at least one TGFβ inhibitor as defined herein, and/or comprising the reprogramming medium of the present invention. In some embodiments, the kit further comprises at least one cardiomyocyte-inducing transcription factor and/or at least one cardiomyocyte-inducing microRNA as defined herein, or an expression vector comprising a nucleotide sequence encoding the transcription factor and/or microRNA. The expression vector is e.g. a viral vector, preferably a lentiviral vector.

In some embodiments of each of the aspects of the present invention, the at least one Tyk2 inhibitor is Baricitinib, and the at least one TGFβ inhibitor is SB43152. In some embodiments of each of the aspects of the present invention, the at least one Tyk2 inhibitor is Ruxolitinib, and the at least one TGFβ inhibitor is TEW-7197. In some embodiments of each of the aspects of the present invention, the at least one Tyk2 inhibitor is Ruxolitinib, and the at least one TGFβ inhibitor is SB43152. In some embodiments of each of the aspects of the present invention, the at least one Tyk2 inhibitor is PF-06826647, and the at least one TGFβ inhibitor is SB43152. In some embodiments of each of the aspects of the present invention, the at least one Tyk2 inhibitor is BMS-986165, and the at least one TGFβ inhibitor is SB43152. In some embodiments of each of the aspects of the present invention, the at least one Tyk2 inhibitor is BMS-986165 and PF-06826647, and the at least one TGFβ inhibitor is SB43152.

### EXAMPLES

To facilitate an understanding of the present invention, a more complete description of the invention will be rendered by reference to specific examples and drawings. Preferred examples of the present invention are shown in the drawings. The present invention may, however, be embodied in many different forms and should not be construed as limited to the examples set forth herein. Rather, these examples are provided so that the present invention will be thorough and complete.

### Materials and Methods

### Lentivirus Preparation and Infection

The lentiviral vector used in this experiment was prepared by co-transfecting human embryonic kidney epithelial cell line HEK 293T with pLL, engineered based on lentiviral vectors PLenti-Lox3.7 (pLL3.7) and FU-tet-o-hOct4, plasmid pVSVg expressing envelope protein vesicular stomatitis virus G protein, plasmid pRSV rev expressing envelope protein to aid in release from the nucleus for shell assembly, and plasmid pMDLg/pRRE carrying envelope and matrix multi-protein expression gene Gag, protease, reverse transcriptase and integrase multi-protein expression gene Pol, and Rev response element RRE for packaging.

Mouse Myocardial Infarction Modeling and Overexpression of Gene In Situ in Myocardial Infarction Area with Lentivirus

1. Anesthesia method: anesthesia was induced with continuous inhalation of isoflurane. Induction concentration: 5%; maintenance concentration: 1%.

2. Endotracheal intubation: prior to intubation, the mouse surgical site was depilated with depilatory cream. The depilation area was under the armpit of its left upper limb, with an area of about 4 square centimeters. The mouse was hung on an inclined cardboard by hanging upper incisor teeth of the mouse with a thin thread. The mouse's throat was illuminated using a gooseneck light. At this point the tongue of the mouse was gently pulled and the trachea was visible.Endotracheal intubation was performed using an indwelling needle. After successful intubation, the mouse was removed from the cardboard and secured to an operating table with medical tape to allow connection to a ventilator while maintaining anesthesia. While maintaining anesthesia, the tidal volume was set to 200 μL/min.

3. The virus was taken out from a −80° C. refrigerator in advance and thawed on ice. After thawing, the virus liquid was gently mixed using a pipette. When lentiviruse with FU-tet-o vector was used, an equal volume of FUdeltaGW-rtTA was pre-mixed (e.g. FU-tet-o-EGFP 50 μL and FUdeltaGW-rtTA 50 μL were mixed). The concentrated virus was aspirated into a microsyringe (using a 27 g needle) in advance and kept on ice until use.

4. Operation method: the mouse was in the right recumbent position. The skin was cut vertically approximately 2 mm below the left axilla of the mouse and a purse-string knot was placed in the wound after the incision. The mouse skin and muscle were bluntly separated, and the pectoralis major muscle was further bluntly separated, exposing the left 4th-5th intercostal space, where the chest was entered. The left ventricle was observed by gentle placement of a thoracotome and slow expansion of the wound. Since the mouse phrenic nerve is very thin, care was taken not to deliberately dissociate the entire pericardium. The left anterior descending coronary artery was observed under a stereomicroscope by gently tearing a little pericardium below the junction of the main pulmonary artery and the left atrial appendage. The proximal end was ligated using a sliding wire. Color changes in the anterior wall of the ventricle were observed after ligation, and the anterior wall of the left ventricle immediately turned pale with transient ventricular arrhythmias. At this time, concentrated virus could be injected.

5 Immediately after ligation of the left anterior descending coronary artery, concentrated virus injection was performed at the edge of the ischemic pale area, 2-3 injections, total volume of concentrated virus: 60 μL. Upon injection, significant bleaching of the myocardium at the injection site was observed. When there was bleeding in the myocardium, medical absorbent cotton was gently pressed until bleeding stopped.

6. The purse-string knot was tightened to close the chest. The isoflurane anesthesia canister was closed. A 10 ml syringe was inserted between the thorax and muscle of the mouse. The mouse thorax was slowly squeezed to expel the gas into the space of thorax and muscle of the mouse. Air was aspirated at this point to avoid pneumothorax complications in the mouse.

7. The mouse was placed on a 42° C. warm bed and would recover after a few minutes.

8. When lentivirus with FU-tet-o vector was used, drinking water of the mouse was replaced with a solution containing 1 mg/mL Doxycycline hyclate and 2 mg/ml sucrose the day after surgery to induce sustained expression of the fragment of interest.

In the heart tube, the supernatant was sucked off, and a white precipitate was observed at the bottom of the cannula. The precipitate was resuspended with precooled PBS, with resuspension ratio of virus-containing medium: PBS=525:1. The concentrated virus after resuspension was dispensed into 50 μL/tube, cryopreserved in a −80° C. refrigerator, and taken as needed.

### Administration of Small Molecule Drugs in Mice

The small molecule SB431542 and Baricitinib were co-dissolved in DMSO to prepare a stock solution, in which the concentration of both small molecules being 100 mg/ml, which was stored in a −80° C. refrigerator. Prior to drug injection, the small molecule stock solution was dissolved in a drug delivery solvent, which was now used and prepared. 2C was administered at a dose of 5 mg/kg/d by intraperitoneal injection. Solvent formulation: 5% Tween-80, 30% PEG300, and 65% deionized water.

### Section Freezing and Immunofluorescence Staining

1. The mouse was killed by cervical approach and the heart was taken out; The heart was dissected coronally along the ligation point and the apical myocardial infarction area was obtained. The blood in the heart was squeezed out in PBS as much as possible. The heart was placed in 4% paraformaldehyde at 4° C. for 3.5 hours; rinsed with PBS; and then dehydrated in 30% sucrose at 4° C. overnight.

2. The tissue was embedded with O.C.T. and frozen in liquid nitrogen.

3. Temperature of a slicing cabinet and temperature of a cutter head were both set to −22° C. The embedded block was firstly trimmed, and then frozen sliced with a thickness of 10 μm. The section was fixed with acetone at 4° C. for 5 minutes and dried in the dark at room temperature to prevent peeling.

4. For fragile antigen, the section may be soaked in citrate buffer at 37° C. for 20 min. The section was washed three times with TBS-Tween20 for 10 min each.

5. 0.3% Triton-100 TBS-Tween20 solution 10 min×3 times at 37° C. (30% stock solution was first prepared: Triton x-100 28.2 ml+TBS-Tween20 72.8 ml, placed in a 37° C. water bath for 2-3 hours to fully dissolve, and then diluted when used); the section was rinsed with TBS-Tween20 for 5 min×3 times.

6. 10% NDS 2% BSA were dissolved in 0.3% Triton-100 TBS-Tween20 solution for blocking for 30 min.

7. The blocking solution was thrown away, and primary antibody (diluted in 10% NDS and 2% BSA according to the proportion recommended in the instruction) was added, and placed at 4° C. for overnight.

8. The section was washed three times with TBS-Tween20 for 5 min each.

9. Secondary antibody (1:1000 diluted in 2% BSA in TBS) was added and left for 1 hour at room temperature in the dark.

10. The section was washed three times with TBS-Tween20 for 5 min each.

11. The section was mounted with anti-quenching mounting medium containing DAPI stain and observed under a laser confocal microscope or protected from light.

12. Laser confocal microscope image acquisition.

13. Imagej software (NIH) assisted analysis.

### Masson Staining

The section was routinely dewaxed to water; stained with the prepared Weigert iron hematoxylin staining solution for 5 min to 10 min; differentiated with an acidic ethanol differentiation solution for 5-15 s, and washed with water; blued with Masson bluing solution for 3-5 min, and washed with water; washed with distilled water for 1 min; stained with ponceau magenta staining solution for 5-10 min; (during the above operation, a weak acid working solution was prepared according to a ratio of distilled water:weak acid solution=2:1), washed with weak acid working solution for 1 min; washed with phosphomolybdic acid solution for 1-2 min; washed with the prepared weak acid working solution for 1 min; directly put into aniline blue staining solution for staining for 1-2 min; washed with the prepared weak acid working solution for 1 min; quickly dehydrated with 95% ethanol; dehydrated with anhydrous ethanol for 3 times, 5-10 s each time; cleared with xylene for 3 times, 1-2 min each time; andmounted with neutral gum.

### Echocardiography in Mouse

Anesthesia method: anesthesia was induced with continuous inhalation of isoflurane. Induction concentration: 5%; maintenance concentration: 1%. Mouse chest was depilated. Image acquisition was performed using a Vevo 2100 (VisualSonics) small animal ultrasound system.

### Method for Reprogramming Induced Cardiomyocytes:

NSFs (neonatal mouse skin fibroblasts) were isolated from mouse skin 1 day after birth and digested with collagenase.P0 was cryopreserved and P1 was resuscitated for induction.nCFs (neonatal mouse cardiac fibroblasts) were isolated from mouse heart 1 day after birth, digested with collagenase and plated in a 10 cm culture dish. Residual cardiomyocytes were removed with CD90.2 MACS, and the sorted CFs were used for induction.

Human fibroblasts, from ATCC, ˜P8-10 were used for induction.

Reprogramming steps: at d-2, plating cells; at d-1, infecting with virus; andat d0, removing virus-containing medium, and replacing with reprogramming medium. Beating cell count and immunofluorescence assay were performed after approximately 3 weeks.

### MEF Separation

MEF Medium: high glucose Dulbecco's modified Eagle medium (DMEM) supplemented with 10% fetal bovine serum (FBS), 1% GlutaMAX, 1% non-essential amino acids (NEAA) and 1% Pen Strep.

Mouse embryonic fibroblasts (MEFs) were isolated from ICR mouse embryo. Briefly, after removal of head, limbs and viscera, E13.5 embryo was minced with scissors and dissociated in trypsin-EDTA at 37° C. for 10 min. After addition of MEF medium and centrifugation, MEF cells were collected and cultured.

### NSF Separation

NSF Medium: high glucose Dulbecco's modified Eagle medium (DMEM) supplemented with 10% fetal bovine serum (FBS), 1% GlutaMAX, 1% non-essential amino acids (NEAA) and 1% penicillin-streptomycin.

Neonatal mouse skin fibroblasts (NSFs) were isolated from ICR mouse 1 day after birth. Briefly, after sacrifice, the skin was stripped and placed in a PBS solution containing 0.25% Trypsin and digested overnight at 4° C. The next day, the digested skin tissue was taken out and the epidermis was carefully removed. Dermal tissue was cut into pieces, placed in collagenase type I+DNase I (dissolved in MEF medium) for digestion for ˜30 minutes, centrifuged to take hair follicle cells, and skin fibroblasts were collected.

### nCF Separation

nCF Medium: IMDM, supplemented with 20% fetal bovine serum (FBS) and 1% penicillin-streptomycin.

Neonatal mouse cardiac fibroblasts (nCFs) were isolated from ICR mouse 1 day after birth. Briefly, after sacrifice, heart was taken out, chopped and placed in collagenase type II+DNase I (dissolved in nCF medium), 1 mg/ml. After every 5 minutes of digestion, the supernatant of the digestive fluid was collected, and the digested cells were collected by centrifugation until the tissue block was completely digested, and nCFs were collected and cultured.

### nCF Magnetic Separation

MACS buffer: 500 ml PBS, added with 2.5 g BSA, 2 ml EDTA (0.5M), filtered through a 0.22μm filter, and stored at 4° C.

nCFs were digested with Trypsin-EDTA. The cells were collected, resuspended in MACS buffer, added with Thy1.2 magnetic beads and incubated at 4° C. for 30 minutes. The incubated cells were rinsed with MACS buffer, resuspended with MACS buffer, and passed through equilibrated LS column. After 3-4 times of rinsing, cells bound to magnetic beads were collected, counted, and set aside for use.

### Lentiviral Packaging

293T medium: high glucose Dulbecco's modified Eagle medium (DMEM) supplemented with 10% fetal bovine serum (FBS), stored at 4° C.

2×HBS: 500 ml HEPES buffer (50 mM)+280 mM NaCl+10 mM KCl+1.5M Na2HPO4+12 mM Glucose, adjusted to pH 7.05, filtered through a 0.22 μm filter, and stored in a −20° C. refrigerator.

2.5 M CaCl₂: CaCl₂ was dissolved in ddH₂O at 2.5M, filtered through a 0.22 μm filter, and stored in a −20° C. refrigerator.

At D-1, 293T was plated in a 10 cm culture dish. At D0, when the cell confluence was ˜70%, fresh medium was replaced. Target plasmid (15 μg) and three packaging plasmids pMDLg/pRRE, RSV/Rev and VSV-G (5 μg each)+50 μl 2.5 M CaCl₂ were premixed, supplemented with ddH₂O to 500 after mixing well, slowly dropped into 500 μl 2×HBS, shaken to mix well, and dropped into the culture dish, which was shaken gently.12 hours after transfection, fresh medium was replaced, and 48 hours after transfection, virus-containing medium was collected, filtered through a 0.45 μm filter, aliquoted and stored in a −80° C. refrigerator.

Production of iCM from Fibroblasts

iCM reprogramming medium: DMEM/M199 (4:1) supplemented with 10% KnockOut serum replacement (KSR), 10% FBS, 1% GlutaMAX, 1% MEM NEAA, 1% Pen Strep, 2 μg/ml Dox and small molecule mixture 2C (2 μM SB431542, 2 μM Baricitinib).

At D-2, 24-well plate was coated with 0.1% gelatin and then placed in a cell culture incubator at 37° C. for 30 min, then the gelatin was aspirated, and the 24-well plate was inoculated with 80,000 cells per well. At D-1, MEF medium containing 6 ng/μl polybrene was replaced, the cells were infected with FU-tet-o-Gata4, FU-tet-o-Mef2c, FU-tet-o-Tbx5, FUdeltaGW-rtTA, 200 μl unconcentrated virus per well per virus. At D0, the medium was replaced with iCM reprogramming medium, and replaced every 3-4 days.

### Reagent Supplier and Article No.

| | Reagent Name | Supplier | Article No. |
|---|---|---|---|
| 1 | DMEM, high glucose | HyClone | SH30022.01 |
| 2 | MEDIUM 199 | Gibco | 11150-059 |
| 3 | KnockOut Serum | Thermo Fisher | A3181502 |
| | Replacement (KSR) | | |
| 4 | Fetal Bovine Serum (FBS) | VISTECH | SE100-011 |
| 5 | GlutaMAX | Gibco | 35050-061 |
| 6 | MEM NEAA | Gibco | 11140-050 |
| 7 | Pen Strep | Gibco | 15140-122 |
| 8 | Doxycycline hyclate | Sigma-Aldrich | D9891 |
| 9 | SB431542 | selleck | S1067 |
| 10 | Baricitinib | selleck | S2851 |
| 11 | IMDM | Gibco | 12440-053 |
| 12 | Collagenase I | Gibco | 17018029 |
| 13 | Collagenase II | Gibco | 17101015 |
| 14 | Polybrene | Sigma-Aldrich | H9268 |
| 15 | CaCl₂ | Sigma-Aldrich | C7902 |
| 16 | HEPES | Sigma-Aldrich | H4034 |
| 17 | NaCl | Sigma-Aldrich | BP358-212 |
| 18 | KCl | Sigma-Aldrich | P5405 |
| 19 | Na2HPO4 | Sigma-Aldrich | S5136 |
| 20 | Glucose | Sigma-Aldrich | G7021 |
| 21 | Gelatin | Sigma-Aldrich | G9391 |
| 22 | PBS | Corning | 21-040-CVR |
| 23 | Trypsin | Gibco | 15090-046 |
| 24 | Trypsin-EDTA | Gibco | 25200-056 |
| 25 | 0.22 μm filter | Millipore | SLGP033RB |
| 26 | 0.45 μm filter | Millipore | SLHP033RB |
| 27 | 24-well plates | Corning | 353047 |
| 28 | 10 cm Tissue culture | Corning | 353003 |
| | dishes | | |

### Example 1, SB431542 and Baricitinib Promote GMT-Mediated Reprogramming Induced Cardiomyocytes in Mice

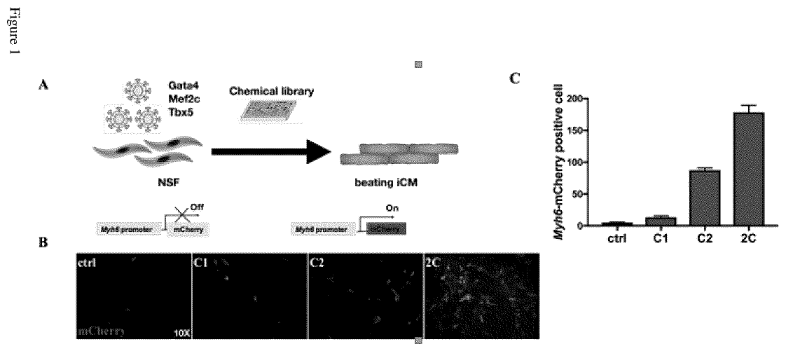

Screening was performed on Myh6-mCherry neonatal mouse dermal fibroblasts. Small molecule library was screened simultaneously with transfection of three genes of GMT (Gata4, Mef2c and Tbx5) to quantify the effect of small molecules on reprogramming by observing the ratio and brightness of reporter genes, as shown in FIG. 1A. Specifically, at D-2 (day-2), Myh6-mCherry neonatal mouse skin fibroblasts (NSF) were plated; at D-1 (day-1), the cells were infected with three lentiviruses expressing Gata4, Mef2c and Tbx5 (GMT), respectively; and at D0 (day 0), the media were replaced with reprogramming media added with small molecules, and replaced every 3-4 days. After three weeks of induction, the number of Myh6-mCherry positive cells was observed and counted to screen for small molecules that promoted the induced cardiomyocyte like cells (iCM).

Through screening, two small molecules, SB431542 (also referred to herein as C1) and Baricitinib (also referred to herein as C2), were found to increase the efficiency of reprogramming induced cardiomyocytes, respectively, and the two small molecules had synergistic effect (their combination is referred to as 2C). See FIG. 1B and 1C.

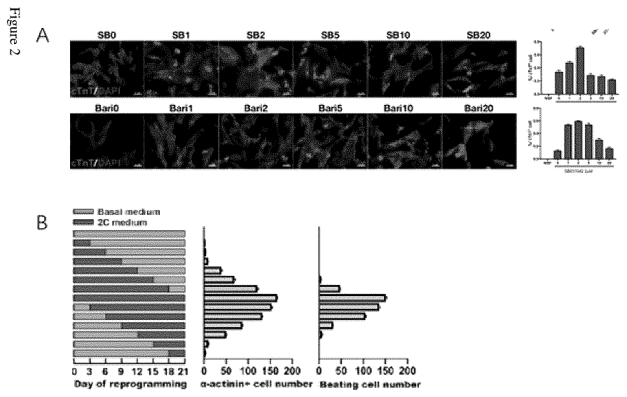

The optimal action concentration and optimal action time of 2C were then further investigated. Specifically, for the optimal action concentration, at D-2, Myh6-mCherry neonatal mouse dermal fibroblasts were plated; at D-1, the cells were infected with three lentiviruses expressing Gata4, Mef2c and Tbx5 (GMT); and at D-0, the media were replaced with reprogramming media containing different combinations of small molecules at different concentrations, and replaced every 3-4 days. First, the optimal action concentration of SB431542 was determined by cardiomyocyte marker cTnT staining after adding different concentrations of SB431542 based on 2 μM Baricitinib. Then the optimal action concentration of Baricitinib was determined by cTnT staining after adding different concentrations of Baricitinib based on 2 μM SB431542.As shown in FIG. 2A, the optimal action concentration of SB431542 was 2 μM and that of Baricitinib was also 2 μM.

For the optimal action time, at D-2, wild-type mouse cardiac fibroblasts (neonatal mouse cardiac fibroblast, nCF) were plated, and residual cardiomyocytes were removed with CD90.2 MACS; at D-1, the cells were infected with three lentiviruses expressing Gata4, Mef2c and Tbx5 (GMT); andat D0, the media were replaced with reprogramming media: Basal medium and 2C medium (i.e. Basal medium+2 μM SB431542+2 μM Baricitinib), and replaced every 3 days.The number of beating iCM cells and the number of cells positive for α-actinin staining were counted at D21.As shown in FIG. 2B, the optimal action time for the combination of the two small molecules was D0-D21 throughout.

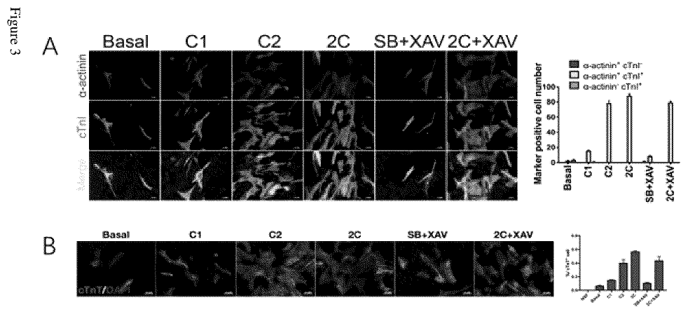

It has been previously reported that the small molecule combination SB431542+XAV939 (Circulation, 2017) can promote reprogramming induced cardiomyocytes. Therefore, the inventors further compared the efficiency of 2C and SB431542+XAV939. Specifically, at D-2, Myh6-mCherry neonatal mouse dermal fibroblasts were plated; at D-1, the cells were infected with three lentiviruses expressing Gata4, Mef2c and Tbx5 (GMT);at D-0, the media were replaced with reprogramming media containing different combinations of small molecules, and replaced every 3-4 days; and at D21, immunofluorescence staining for cTnI, cTnT, α-actinin was performed. The reprogramming media used were: Basal medium, Basal medium+2 μM SB431542 (C1), Basal medium+2 μM Baricitinib (C2), Basal medium+2 μM SB431542+2 μM Baricitinib (2C), Basal medium+2.6 μM SB431542+5 μM XAV939 (SB+XAV), Basal medium+2 μM SB431542+2 μM Baricitinib+5 μM XAV939 (2C+XAV), respectively. As shown in FIG. 3, 2C promoted iCM with a significantly higher efficiency than SB431542+XAV939.

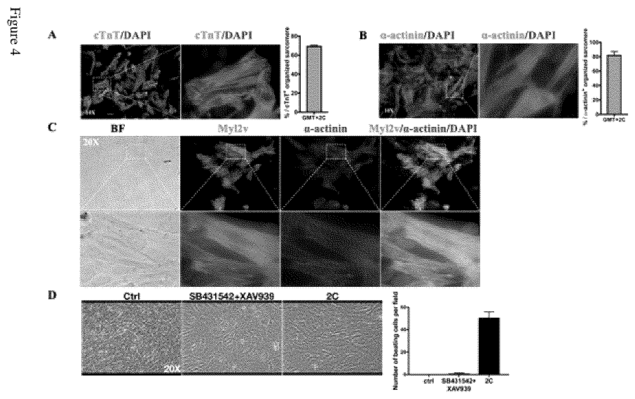

Furthermore, the inventors have found that 2C can not only improve the efficiency of reprogramming induced cardiomyocytes, but also significantly improve the quality of induced reprogramming Specifically, at D-2, Myh6-mCherry neonatal mouse dermal fibroblasts were plated; at D-1, the cells were infected with three lentiviruses expressing Gata4, Mef2c and Tbx5 (GMT); and at D-0, the medium was replaced with reprogramming medium containing 2C, and replaced every 3-4 days; and the expression of cardiac markers or cardiomyocyte phenotype were detected at the 3rd or 4th week. The results are shown in FIG. 4. FIGS. 4A and B show that highly efficient induction of GMT+2C resulted in cardiomyocyte-like cells with 70.1% (4 weeks) cTnT-positive cells and 82.6% (4 weeks) α-actinin-positive cells, all with aligned sarcomere structures. FIG. 4C shows that on skin cells, the vast majority of cardiomyocyte-like cells obtained from GMT+2C induction (4 weeks) were capable of expressing a marker for ventricular-type cardiomyocytes, Myl2v (Myosin light chain 2), demonstrating that iCM obtained from induction is a ventricular subtype of cardiomyocytes. FIG. 4D shows that GMT+2C (3 weeks) very efficiently promoted beating of iCM, demonstrating that induced cardiomyocytes are functionally mature iCM.

### Example 2, 2C in Combination with MT Achieves Reprogramming Induced Cardiomyocytes in Mice

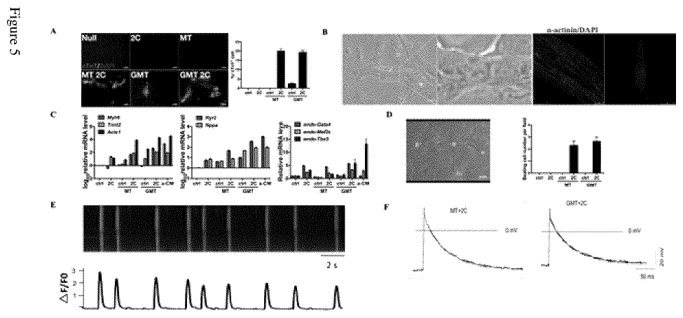

The inventors have surprisingly found that 2C can subtract Gata4 out of the three transcription factors GMT without reducing reprogramming efficiency and quality. Specifically, at D-2, WT mouse cardiac fibroblasts (neonatal mouse cardiac fibroblast, nCF) were plated, and residual cardiomyocytes were removed with CD90.2 MACS; at D-1, treatment was divided into three types: no infection with virus (null), infection with virus expressing Gata4+Mef2c+Tbx5 (GMT), or infection with virus expressing Mef2c+Tbx5 (MT); at D0, the media were replaced with reprogramming media: Basal medium and 2C medium (i.e. Basal medium+2 μM SB431542+2 μM Baricitinib), and replaced every 3 days; and marker expression, myocardial associated gene expression, beating cell count, cellular calcium transients, cellular action potentials, etc. were examined at specified time. The results are shown in FIG. 5.

FIG. 5A shows that 2C could subtract Gata4 from the three GMT genes by cTnT staining at the 3rd week. FIG. 5B shows that cells obtained from MT+2C induction (4 weeks) had an aligned sarcomere structure. FIG. 5C shows that 2C promoted expression of cardiomyocyte genes (cardiomyocyte structural genes, cardiomyocyte function-related genes, cardiomyocyte endogenous transcription factors). FIG. 5D shows that MT+2C induction (3 weeks) resulted in beating functional cardiomyocytes. FIG. 5E shows that functional cardiomyocytes obtained by MT+2C induction (4 weeks) had spontaneous calcium transients. FIG. 5F shows that functional cardiomyocytes obtained from MT+2C induction (6 weeks) had action potentials similar to mature ventricular-type cardiomyocytes.

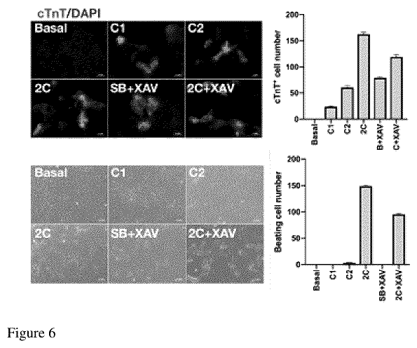

Furthermore, the inventors have further found that subtracting GATA4 from the three GMT genes requires the joint action of 2C. Specifically, at D-2, WT mouse cardiac fibroblasts (neonatal mouse cardiac fibroblast, nCF) were plated, and residual cardiomyocytes were removed with CD90.2 MACS; at D-1, the cells were infected with virus expressing Mef2c+Tbx5 (MT); at D0, the media were replaced with reprogramming media, and replaced every 3 days; and after 3 weeks, cTnT positive cells and beating cells were examined.The reprogramming media were sequentially: Basal medium, Basal medium+2 μM SB431542 (C1), Basal medium+2 μM Baricitinib (C2), Basal medium+2 μM SB431542+2 μM Baricitinib (2C), Basal medium+2.6 μM SB431542+5 μM XAV939 (SB+XAV), Basal medium+2 μM SB431542+2 μM Baricitinib+5 μM XAV939 (2C+XAV). As shown in FIG. 6, upon transduction of MT only, beating cells could be induced only by adding 2C at the same time.

### Example 3, Mechanism of 2C Promoting Reprogramming Induced Cardiomyocytes

The inventors further studied the expression profile of cardiomyocytes induced by reprogramming with MT+2C and GMT+2C by RNA-seq. Specifically, at D-2, WT mouse cardiac fibroblasts (neonatal mouse cardiac fibroblast, nCF) were plated, and residual cardiomyocytes were removed with CD90.2 MACS; at D-1, treatment was divided into three types: no infection with virus (null), infection with virus expressing Gata4+Mef2c+Tbx5 (GMT), or infection with virus expressing Mef2c+Tbx5 (MT); at D0, the media were replaced with reprogramming media, and replaced every 3 days; and after 6 weeks, cells were harvested and total RNA was extracted for RNA-seq.The media used were: Basal medium, Basal medium+2 μM SB431542 (SB), Basal medium+2 μM SB431542+2 μM Baricitinib (2C), Basal medium+2.6 μM SB431542+5 μM XAV939 (SBXAV), respectively. Neonatal CM represents 1-day-old mouse cardiomyocyte and Adult CM represents 8-week-old adult mouse ventricular cardiomyocyte.

As shown in FIG. 7, the data of RNA-seq showed that MT+2C and GMT+2C could be clearly distinguished from other combinations and had a more similar expression profile to adult cardiomyocytes; and compared with other combinations, MT+2C and GMT+2C could induce myocardial specific gene expression and inhibit fibroblast-related gene expression better.

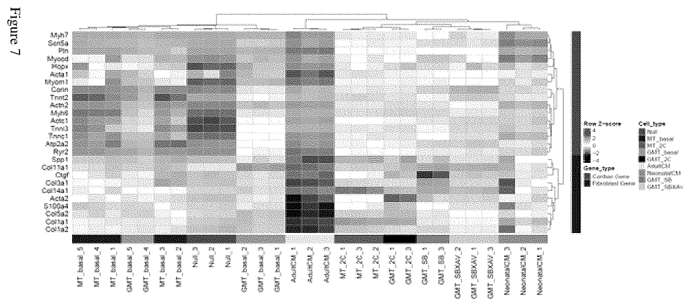

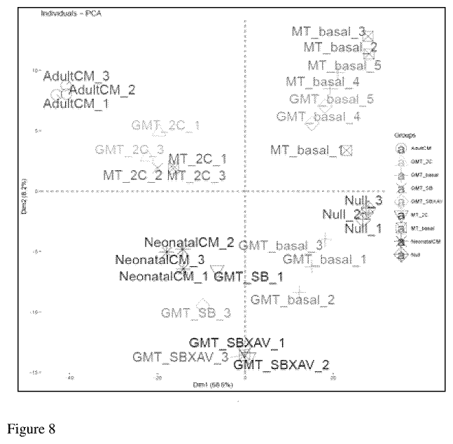

Based on RNA-seq data, principal component analysis of 782 genes with the greatest difference between adult cardiomyocytes and mouse cardiac fibroblasts showed that MT+2C and GMT+2C were closer to adult cardiomyocytes than other published reprogramming methods, as shown in FIG. 8.

Furthermore, to further investigate the role of 2C, GO analysis was performed for genes that were significantly up-regulated and down-regulated with GMT+2C vs GMT. As shown in FIG. 9, 2C significantly up-regulated genes associated with myocardial development and muscle beating, and significantly down-regulated genes associated with cell activation and adhesion.

### Example 4, Substitute for 2C

This example aims to investigate whether SB431542 or Baricitinib can be replaced by small molecules of the same signaling pathway.

Specifically, at D-2 (day-2), Myh6-mCherry neonatal mouse skin fibroblasts (NSF) were plated; at D-1 (day-1),the cells were infected with three lentiviruses expressing Gata4, Mef2c and Tbx5 (GMT), respectively; at D0 (day 0), the media were replaced with reprogramming media added with small molecules, and replaced every 3-4 days; andat D18, beating cells were counted. In addition, at D-2, WT mouse cardiac fibroblasts (nCFs) were plated, and residual cardiomyocytes were removed with CD90.2 MACS; at D-1, the cells were infected with virus expressing Mef2c+Tbx5 (MT); at D0, the media were replaced with reprogramming media added with small molecules, and replaced every 3 days; and after four weeks, cTnT positive cells were counted.2C was used as a positive control.

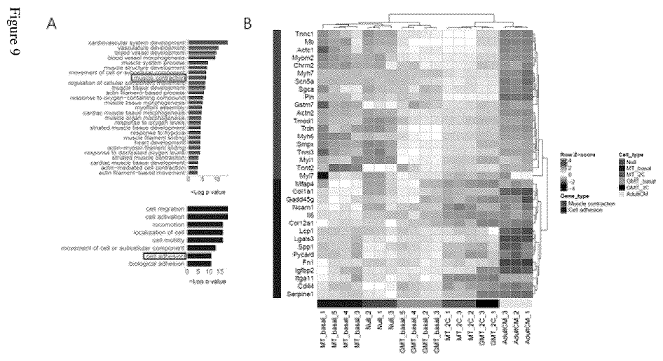

SB431542 is an inhibitor of TGFβ signaling pathway, and this example analyzed whether other small molecules of this signaling pathway could be combined with Baricitinib, including RepSox, GW788388, SD-208, LY364947, Y-27632, LDN-193189, LY2109761 and Galunisertib. Baricitinib is an inhibitor of Jak pathway, and this example analyzed whether other small molecules of this signaling pathway could be combined with SB431542, including Filgotinib, WP1066, Gandotinib, Ruxolitinib and AZD1480. As shown in FIG. 10, SB431542 could be effectively replaced by small molecules of the same signaling pathway, either by beating or by the number of cTnT-positive cells, while for small molecules of the same signaling pathway of Baricitinib, only its structural analogue Ruxolitinib could be used for replacement. It can be seen that the TGFβ signaling pathway is important for reprogramming into cardiomyocytes; however, at the same time, Baricitinib cannot be replaced by most other compounds targeting Jak-Stat.

In addition, some small anti-inflammatory molecules have been reported to increase reprogramming-induced cardiomyocyte efficiency, such as Dexamethasone or Nabumetone. However, as shown in FIG. 11, on the basis of MT transduction, these small anti-inflammatory molecules were used to induce WT mouse cardiac fibroblasts (nCFs) for three weeks in combination with SB431542, respectively, which could not replace the effect of Baricitinib.

As mentioned above, for small molecules of Jak signaling pathway, only its structural analogue Ruxolitinib could be used for replacement.The inventors further examined whether other structural analogs of Baricitinib can effectively replace Baricitinib. As shown in FIG. 12, on the basis of MT transduction, Baricitinib structural analogues were used to induce WT mouse cardiac fibroblasts (nCF) for three weeks in combination with SB431542, respectively, which could replace the effect of Baricitinib.

### Example 5, 2C Promotes Reprogramming Induced Human Cardiomyocytes

The inventors further investigated the role of 2C in transdifferentiation of human fibroblasts to human cardiomyocyte-like cells (human induced cardiomyocyte like cell, hiCM) based on five transcription factors (GATA4, MEF2C, TBX5, MESP1, MYOCD).

Specifically, at D-2, human cardiac fibroblasts were plated; at D-1, the cells were infected with lentivirus expressing GATA4, MEF 2C, TBX5, MESP1, MYOCD (5F); at D0, the media were replaced with reprogramming media: basal/2C medium, and replaced every 3 days; andat the 3rd week, the number of cTnT positive cells was counted, and the phenotype and myocardial related gene expression were detected. Or, at D-2, BJ human epidermal fibroblasts were plated; at D-1, the cells were infected with lentiviruses expressing GATA4, MEF2C, TBX5, MESP1, MYOCD (5F); at D0, the media were replaced with reprogramming media: basal/2C medium, and replaced every 3 days; andat the 3rd week, spontaneous calcium transients were examined

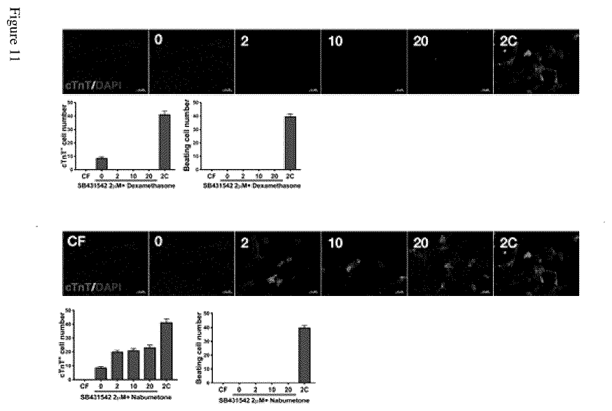

The results are shown in FIG. 13. FIG. 13A shows that 2C very efficiently promoted transdifferentiation of human fibroblasts into hiCMs. FIG. 13B shows that the hiCM obtained by induction had a good sarcomere structure. FIG. 13C shows that 2C significantly increased the expression of cardiac-related genes. FIG. 13D shows that the hiCM obtained by induction had a spontaneous calcium transient.

Five transcription factors (GATA4, MEF2C, TBX5, MESP1, MYOCD) are reported to be necessary for the induction of hiCM. The inventors have surprisingly found that 2C can reduce the number of transcription factors required for reprogramming of human cells without reducing reprogramming efficiency.

Specifically, at D-2, BJ human epidermal fibroblasts were plated; at D-1, the cells were infected with lentiviruses expressing four species (4F) of GATA4, MEF2C, TBX5, MESP1 and MYOCD; at D0, the media were replaced with reprogramming media: basal/2C medium, and replaced every 3 days; and at the 3rd week, the number of a-actinin positive cells was examined.

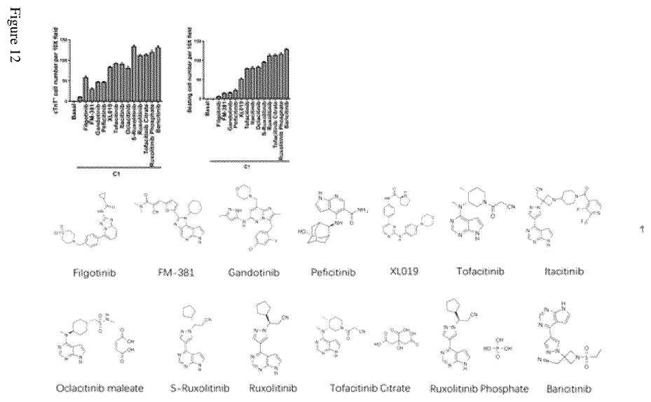

The results are shown in FIG. 14. In the presence of 2C, TBX5 or MESP1 was not essential, in particular MESP1. The efficiency of 2C in combination with GATA4, MEF2C, TBX5, and MYOCD was even higher than that of 2C+5F.

### Example 6, In Vivo In Situ Reprogramming Induced Cardiomyocytes with 2C

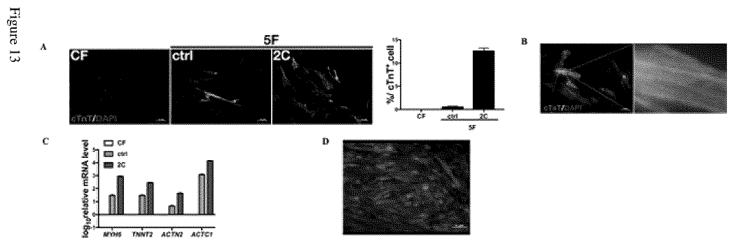

To verify the effect of 2C in in vivo in situ reprogramming, mature lentiviral overexpression system was used to overexpress transcription factors such as Gata4, Mef2c and Tbx5 in situ in the myocardial infarction region of mice, in combination with small molecule compounds to improve the efficiency of transdifferentiation and myocardial infarction treatment effect. In order to better evaluate the conversion efficiency of cell fate, the transgenic mouse model of Postn-MerCreMer and Rosa-loxp-stop-loxp-tdTomato was used, myofibroblasts in myocardial infarction area were traced, the proportion of cardiomyocytes in red fluorescent cells (once cardiac myofibroblasts) was calculated by immunofluorescence of tissue sections combined with laser confocal microscope counting, myocardial infarction area cell separation and other methods, so as to determine the transdifferentiation efficiency of myofibroblasts into myocardium. The therapeutic effect of cardiac repair was evaluated comprehensively by means of echocardiography, ECG monitoring, MRI and animal behavior detection. In vivo reprogramming in situ is shown in FIG. 15.

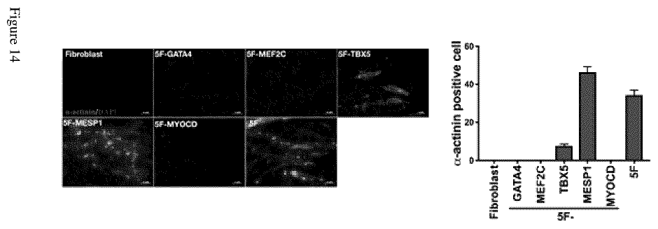

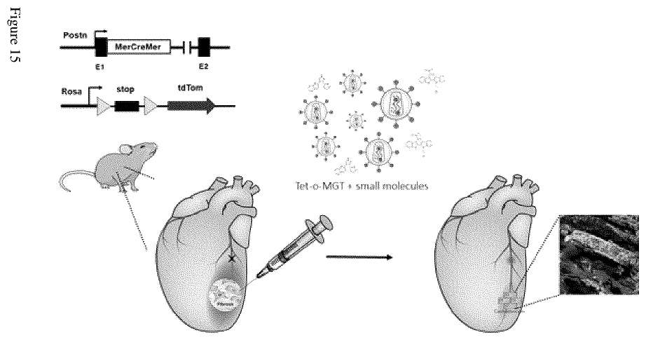

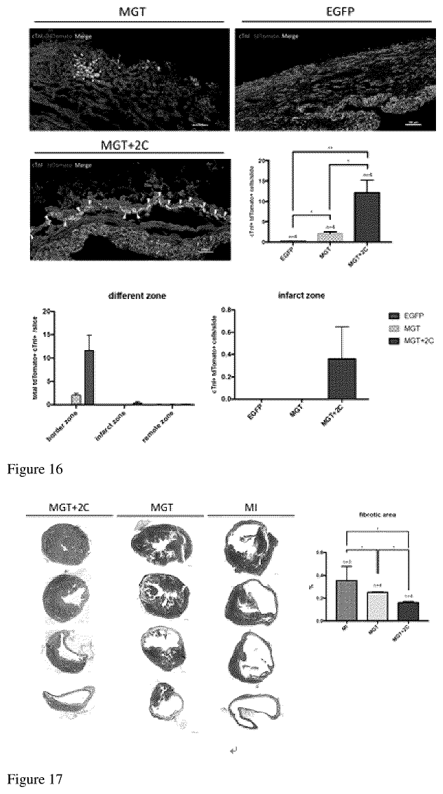

As shown in FIG. 16, in vivo lineage tracing experiment indicates that GMT+2C had better in vivo in situ reprogramming efficiency than GMT. Compared with GMT, GMT+2C had significantly improved reprogramming effect in the edge area and infarct area of myocardial infarction. In addition, through Masson staining, the GMT+2C treatment group was found to have a significant reduction in fibrotic scar area compared to GMT in the myocardial infarction mouse model, as shown in FIG. 17.

More surprisingly, however, in the absence of any transgene, a certain amount of in vivo reprogramming in situ was observed for the neat 2C treatment group and the reprogramming efficiency was comparable to that of the GMT treatment group, as shown in FIG. 18. In addition, Masson staining shows (FIG. 19) that the fibrotic scar area was significantly reduced in the neat 2C treatment group compared to the solvent treatment group. Such results suggest that 2C has the potential to be used alone to treat myocardial infarction.

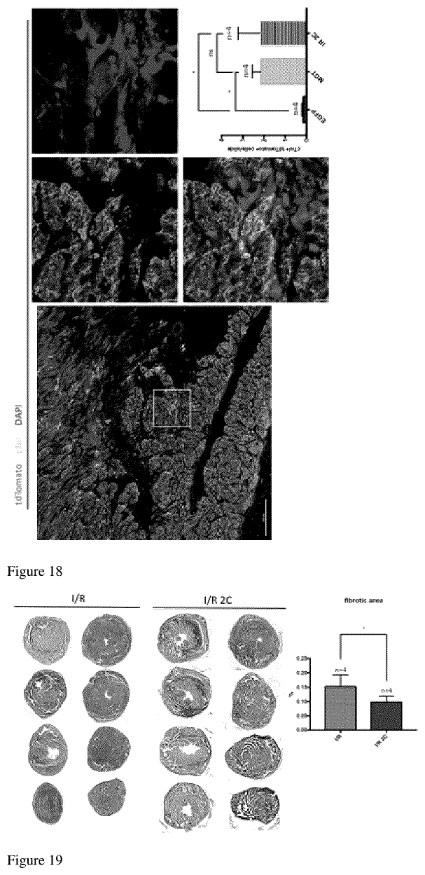

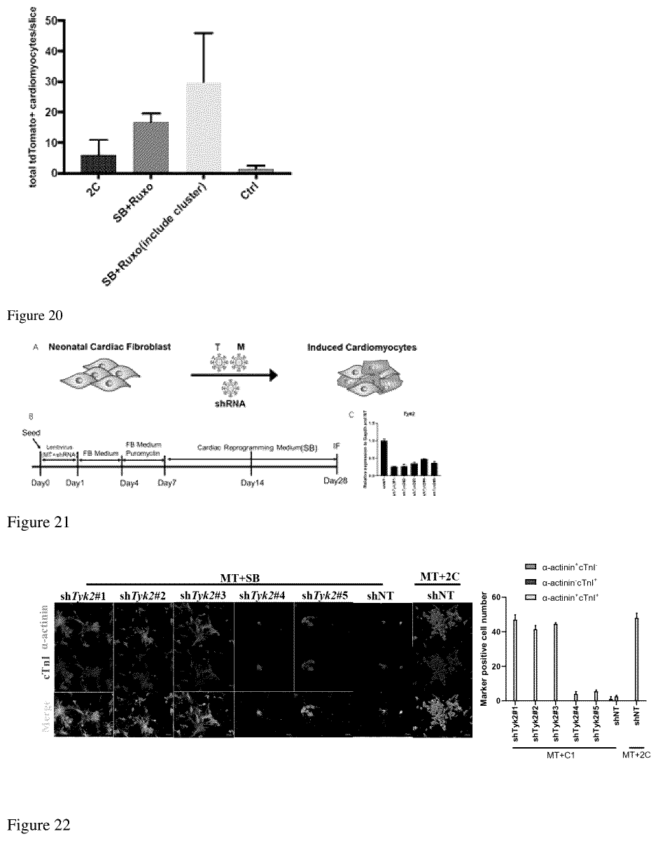

FIG. 20 shows that the combination of Ruxolitinib with SB43152 had a superior effect.

### Example 7, Inhibition of Tyk2 Signaling Pathway Promotes Reprogramming Induced Human Cardiomyocytes

1. Knockdown of Tyk2 by shRNA Affects Cardiomyocyte Reprogramming

As described above, the combination (2C) of the small molecule Baricitinib (C2) and SB43152 (C1) can significantly improve the efficiency of cardiomyocyte reprogramming mediated by the transcription factor combination GMT (Gata4, Mef2c and Tbx5), and the small molecule combination can also reduce the number of exogenous transcription factors required for reprogramming without reducing the efficiency and quality of reprogramming, i.e. high efficiency of cardiomyocyte reprogramming can be achieved using the transcription factor combination MT (Mef2c and Tbx5) in the presence of the small molecule combination. However, as shown above, most of the small molecules of Jak signaling pathway cannot replace the role of Baritinib in myocardial reprogramming Thus, Baricitinib may function through other signal pathways.

The inventors have now surprisingly found that knockdown of Tyk2 expression in cells can replace the effect of Baricitinib (C2).The experimental design of this example is shown in FIG. 21. Briefly, shRNAs targeting Tyk2 gene (shTyk2#1, shTyk2#2, shTyk2#3, shTyk2#4, shTyk2#5) were designed, introduced into neonatal mouse fibroblasts together with transcription factor combination MT, and then induced in reprogramming medium containing C1 to test the efficiency of reprogramming into cardiomyocyte-like cells. FIG. 21C shows that all the five shRNAs could knockdown the expression of Tyk2.

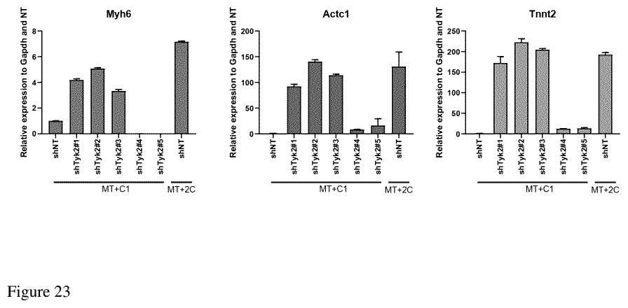

Immunofluorescence detection of cardiac specific markers cTnI and a-actinin showed that shTyk2#1, shTyk2#2, shTyk2#3, combined with transcription factor MT and C1, respectively, could achieve similar efficiency of cardiomyocyte reprogramming as small molecule combination 2C. shNT was a non-targeted control.The results are shown in FIG. 22. In addition, qPCR also demonstrated a significant increase in cardiac-specific marker expression under the condition of MT+C1 and Tyk2 knockdown (FIG. 23).

The sequences of Tyk2-specific shRNAs capable of knocking down Tyk2 and improving the efficiency of myocardial reprogramming are as follows:

shTyk2#1:
(SEQ ID NO: 1)
CCCATCTTCATTAGCTGGGAACTCGAGTTCCCAGCTAATGAAGATGGG;
shTyk2#2:
(SEQ ID NO: 2)
CCCTTCATCAAGCTAAGTGATCTCGAGATCACTTAGCTTGATGAAGGG;
shTyk2#3:
(SEQ ID NO: 3)
CCACTTTAAGAATGAGAGCTTCTCGAGAAGCTCTCATTCTTAAAGTGG.

It can be seen that specific knockdown of Tyk2 can promote cardiomyocyte reprogramming

### 2. Knockout of Tyk2 Through Gene Editing Affects Cardiomyocyte Reprogramming

To further confirm the role of specific inhibition of Tyk2 in cardiomyocyte reprogramming, the inventors further designed five different sgRNAs targeting Tyk2 gene, knocked out the Tyk2 gene of neonatal mouse fibroblasts using CRISPR technique, and tested the efficiency of cardiomyocyte reprogramming in the presence of transcription factor MT and C1, respectively. sgNT was a control for non-targeted Tyk2.

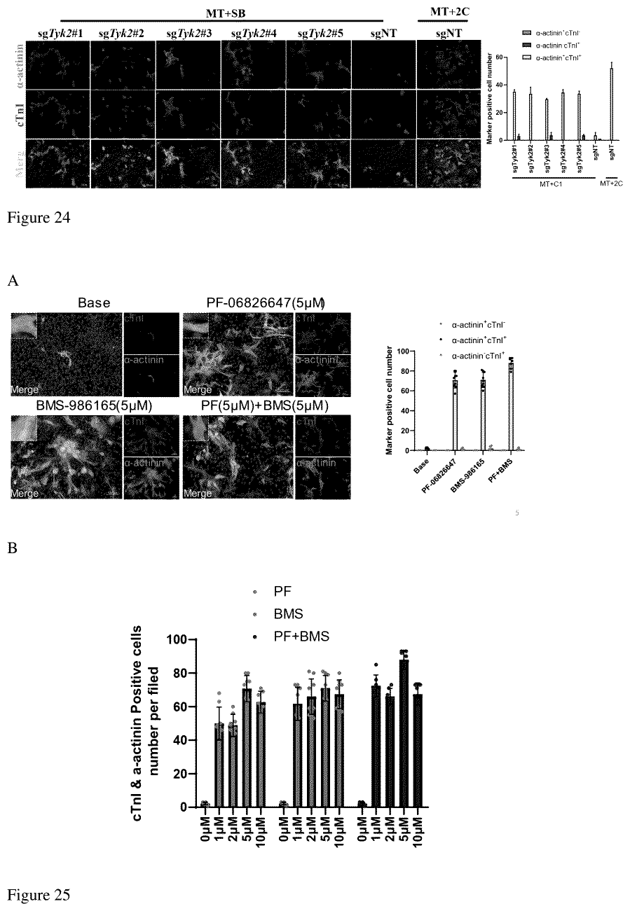

The results are shown in FIG. 24 Immunofluorescence detection of cardiac-specific markers cTnI and a-actinin showed that fibroblasts with Tyk2 gene knocked out could achieve efficient reprogramming of cardiomyocytes in the presence of the transcription factor MT and the small molecule compound C1.

3. Tyk2 Small Molecule Inhibitors Promote Reprogramming of Neonatal Mouse Fibroblasts into Cardiomyocytes

### Isolation of Neonatal Mouse Fibroblasts:

Neonatal suckling mice within 24 h were ordered from Vital River. In an ultraclean bench, heart tissue was taken, cut with sterilized surgical instruments, added with an appropriate amount of Type II Collagenase (1 mg/mL), digested at 37° C. constant temperature, after full digestion, washed twice with IMDM (20% FBS+1% PS+1% NEAA+1% Glu-Max) medium, resuspended with the medium, and plated in a 10 cm culture dish. After 24 h, fresh IMDM was added, and on the fourth day, CD90.2 (anti-Thy1+) was used for MACS separation. The sorted cells were plated in a 24-well plate 2-5×10\hat{ }5/well), and infected with Fu-tet-o-Mef2c-T2A-Tbx5 virus and rtTA 24 h after plating. After 24 h, the medium was replaced with reprogramming medium, and replaced every 3 days. Beating cells could be seen at the 4th week, and a large number of cTnI and a-actinin could be seen by immunofluorescence staining.

### Reprogramming Medium:

10% FBS, 10% KSR, DMEM/M199 [4:1], 1% PS+1% NEAA+1% Glu-Max, 2 uM SB431542, Tyk2 inhibitor (BMS-986165 or PF-06826647), replaced every 3 days. Tyk2 inhibitor was used at 1, 2, 5 and 10 uM, and the optimal concentration is shown in the concentration gradient curve.

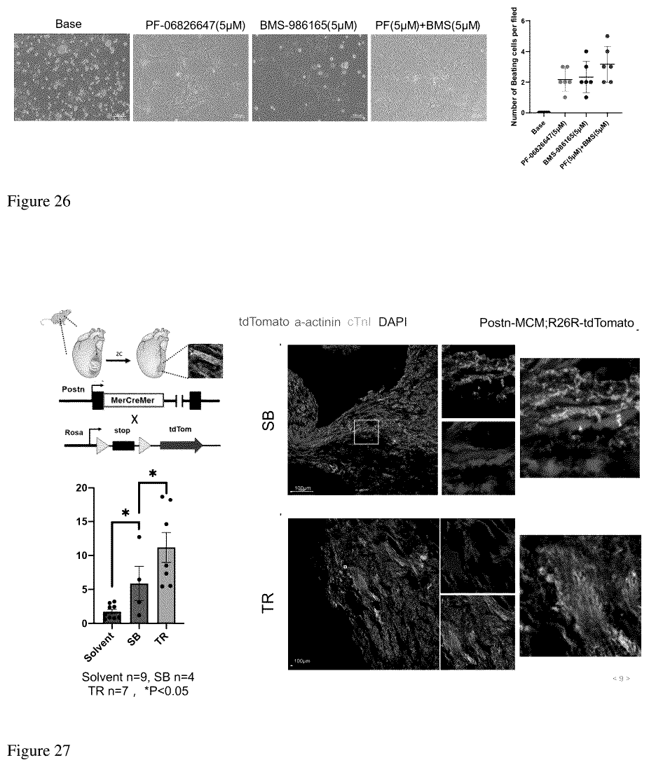

The results are shown in FIGS. 25 and 26. The results show that Tyk2 small molecule inhibitors effectively promoted cardiomyocyte reprogramming.

### Example 8, Replacing Tyk2 Inhibitor and TGFβ Inhibitor Promotes Reprogramming Induced Cardiomyocytes

This example tested the effect of the combination of Ruxolitinib+TEW-7197 on reprogramming induced cardiomyocytes by replacing Baricitinib with another Tyk2 inhibitor, Ruxolitinib, and SB43152 with another TGFβ inhibitor, TEW-7197.

### Mouse MI Surgery and Virus Injection

WT ICR male, 8 w, anesthetized with tribromoethanol and subjected to thoracotomy, heart was squeezed out, left anterior descending coronary artery was ligated, 10 μl concentrated retroviral vector pMX-MGT/pMX-MT was injected, the heart was put back, and the skin was sutured.

### In Vivo Administration

The small molecules Tew-7197 and Ruxolitinib were dissolved in DMSO at a concentration of 200 mM in both small molecule stocks and stored in a −20 degree refrigerator. Prior to drug injection, the small molecule stock solution was dissolved in a drug delivery solvent, which was now used and prepared. Tew-7197 was administered at a dose of 6 mg/kg/d and Ruxolitinib was administered at a dose of 60 mg/kg/d by intraperitoneal injection. Solvent formulation: 5% Tween-80, 30% PEG300, and 65% deionized water. Results were detected five weeks after administration.

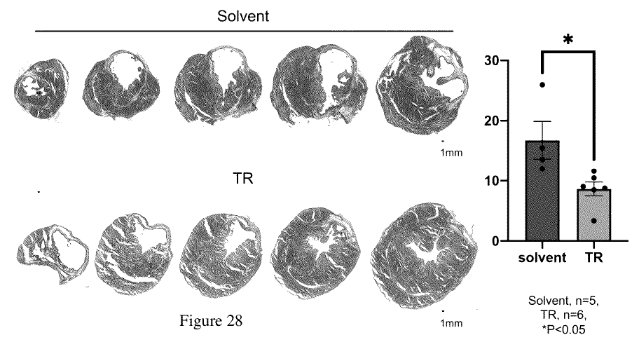

The experimental results are shown in FIGS. 27 and 28, showing that the Tyk2 inhibitor Ruxolitinib and the TGFβ inhibitor TEW-7197 also improved cardiac reprogramming efficiency in situ and improved cardiac fibrosis after MI.

### Example 9, Combination of MYOCD and 2C Improves the Efficiency of hiCM

### Normal Human Cardiac Fibroblast Cell Culture

Cells were purchased from Lonza, Cat. CC2904, and passaged in high glucose Dulbecco's modified Eagle medium (DMEM) supplemented with 10% fetal bovine serum (FBS).

### Lentivirus Packaging

293T medium: high glucose Dulbecco's modified Eagle medium (DMEM) supplemented with 10% fetal bovine serum (FBS), stored at 4° C.

2×HBS: 500 ml HEPES buffer (50 mM)+280 mM NaCl+10 mM KCl+1.5M Na2HPO4+12 mM Glucose, adjusted to pH 7.05, filtered through a 0.22 μm filter, and stored in a −20° C. refrigerator.

2.5 M CaCl₂: CaCl₂ was dissolved in ddH₂O at 2.5M, filtered through a 0.22 μm filter, and stored in a −20° C. refrigerator.

At D-1, 293T was plated in a 10 cm culture dish. At D0, when the cell confluence was ˜70%, fresh medium was replaced. Target plasmid (15 μg) and three packaging plasmids pMDLg/pRRE, RSV/Rev and VSV-G (5 μg each)+50 μl 2.5M CaCl₂ were premixed, supplemented with ddH₂O to 500 μl, after mixing well, slowly dropped into 500 μl of 2×HBS, shaken to mix well, and dropped into the culture dish, which was shaken gently.12 hours after transfection, fresh medium was replaced, and 48 hours after transfection, virus-containing medium was collected, filtered through a 0.45 μm filter, aliquoted and stored in a −80° C. refrigerator.

### Retrovirus Packaging

At D-1, 293T was plated in a 10 cm culture dish. At D0, when the cell confluence was ˜70%, fresh medium was replaced. 8 μg:8 μg:1 μg (retroviral DNA:pUMVC:VSV-G)+50 μl 2.5M CaCl₂ were premixed, supplemented with ddH₂O to 500 μl, after mixing well, slowly dropped into 500 μl of 2×HBS, shaken to mix well, and dropped into the culture dish, which was shaken gently. 12 hours after transfection, fresh medium was replaced. 48 hours after transfection, virus-containing medium was collected, filtered through a 0.45 μm filter, added with 1/5 volume of TransLvTM Lentivirus Precipitation Solution (Transgen, FV101), mixed well, allowed to stand at 4° C. for 40 min, centrifuged at 4° C. for 8000 g, the supernatant was discarded, and the precipitate was resuspended with PBS.

Production of iCM from Fibroblasts

iCM reprogramming medium: DMEM/M199 (4:1) supplemented with 10% KnockOut serum replacement (KSR), 10% FBS, 1% GlutaMAX, 1% MEM NEAA, 1% Pen Strep, 2 μg/ml Dox and small molecule mixture 2C (2 μM SB431542, 2 μM Baricitinib).

At D-2, 24-well plate was coated with 0.1% gelatin and then placed in a cell culture incubator at 37° C. for 30 min, then the gelatin was aspirated, and the 24-well plate was inoculated with 80,000 cells per well. At D-1, MEF medium containing 6 ng/μl polybrene was replaced, the cells were infected with FU-tet-o-MYOCD, FUdeltaGW-rtTA, 200 μl unconcentrated virus per well per virus. At D0, the medium was replaced with iCM reprogramming medium, and replaced every 3-4 days. iCM production was examined after 4 weeks of treatment.

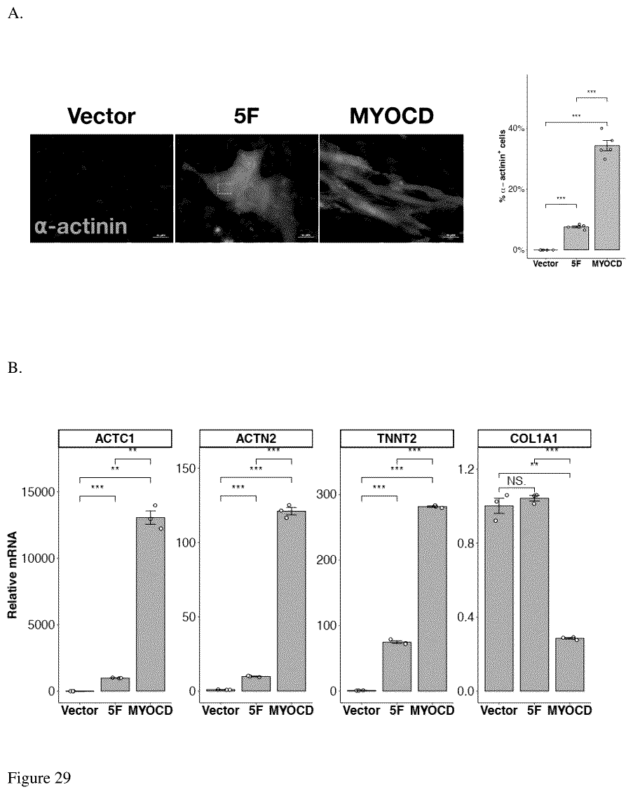

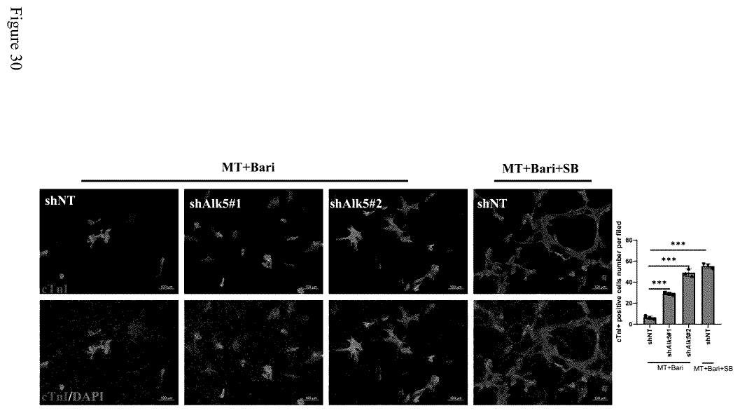

The results are shown in FIG. 29, showing that the combination of MYOCD and 2C achieved significantly improved hiCM induction efficiency.

### Example 10, Knockdown of TGF Receptor Alk5

Isolation of neonatal mouse fibroblasts: Neonatal suckling mice within 24 h were ordered from Vital River. In an ultraclean bench, heart tissue was taken, cut with sterilized surgical instruments, added with an appropriate amount of Type II Collagenase (1 mg/mL), digested at 37° C. constant temperature, after full digestion, washed twice with IMDM (20% FBS+1% PS+1% NEAA+1% Glu-Max) medium, resuspended with the medium, and plated in a 10 cm culture dish. After 24 h, fresh IMDM was added, and on the fourth day, CD90.2 (anti-Thy1+) was used for MACS separation. The sorted cells were plated in a 24-well plate (2-5×10\hat{ }5/well), and infected with Fu-tet-o-Mef2c-T2A-Tbx5 viruses and rtTA 24 h after plating. After 24 h, the medium was replaced with reprogramming medium, and replaced every 3 days. Beating cells could be seen in 4 weeks, and a large number of cTnI and a-actinin could be seen by immunofluorescence staining.

Reprogramming medium: 10% FBS, 10% KSR, DMEM/M199 [4:1], 1% PS+1% NEAA+1% Glu-Max, 2 uM Baricitinib, replaced every 3 days. The concentration of SB431542 in 2C medium was 2 uM.

Alk5 # 1 and # 2 Target Sequences
#1:
CCGGATAGCTGAAATTGACCTAATTCTCGAGAATTAGGTCAATTTCA
GCTATTTTTTG
#2:
CCGGGCTGACAGCTTTGCGAATTAACTCGAGTTAATTCGCAAAGCTG
TCAGCTTTTTG

The results are shown in FIG. 30, showing that after knockdown of Alk5, the combination of MT and Baricitinib was able to produce large numbers of cTnI and a-actinin positive cardiomyocytes in the absence of SB431542.

### Example 11, 2C Improve Cardiac Function

### Retrovirus Packaging

At D-1, 293T was plated in a 10 cm culture dish. At D0, when the cell confluence was ˜70%, fresh medium was replaced. 8 μg:8 μg:1 μg (retroviral DNA:pUMVC:VSV-G)+50 μl 2.5M CaCl₂ were premixed, supplemented with ddH₂O to 500 after mixing well, slowly dropped into 500 μl of 2×HBS, shaken to mix well, and dropped into the culture dish, which was shaken gently. 12 hours after transfection, fresh medium was replaced. 48 hours after transfection, virus-containing medium was collected, filtered through a 0.45 μm filter, added with 1/5 volume of TransLvTM Lentivirus Precipitation Solution (Transgen, FV101), mixed well, allowed to stand at 4° C. for 40 min, centrifuged at 4° C. for 8000 g, the supernatant was discarded, and the precipitate was resuspended with PBS.

### Mouse MI Surgery and Virus Injection

WT ICR male, 8 w, anesthetized with tribromoethanol and subjected to thoracotomy, heart was squeezed out, left anterior descending coronary artery was ligated, 10 μl concentrated pMX-MGT/pMX-MT was injected, the heart was put back, and the skin was sutured.

### In Vivo Administration

2C were respectively dissolved in DMSO and stored at −20° C. Before each administration, the stock solution was dissolved in cosolvent (30% PEG+5% Tween80 ddH2O), C1 10 mg/kg/d, C2 20 mk/kg/d, and injected intraperitoneally.

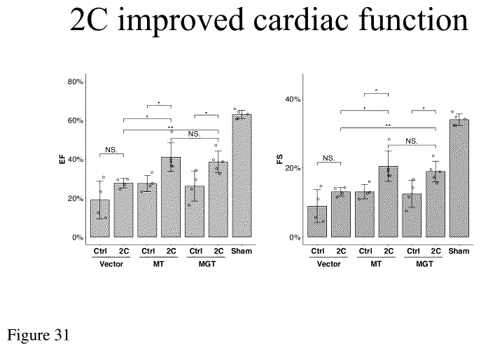

The experimental results are shown in FIG. 31, showing that 2C could improve cardiac function in the heart.

1. A method for reprogramming a starting cell into a cardiomyocyte, the method comprising contacting the starting cell with at least one Tyk2 inhibitor and/or at least one TGFβ inhibitor.

2. The method of claim 1, wherein the Tyk2 inhibitor is selected from the group consisting of Baricinib, Ruxolitinib, S-Ruxolitinib, Tofacitinib, Ocacitinib maleate, Itacitinib, Peficitinib, Gandotinib, FM-381, Filgotinib, PF-06826647, BMS-986165, or a structural analog thereof.

3. The method of claim 1, wherein the TGFβ inhibitor is selected from the group consisting of SB43152, TEW-7197, RepSox, GW788388, SD-208, LY364947, Y-27632, LDN-193189, LY2109761, and Galunisertib, or a structural analog thereof.

4. The method of claim 1, wherein the Tyk2 inhibitor has a concentration of about 0.1 μM to about 50 μM, preferably about 2 μM.

5. The method of claim 1, wherein the TGFβ inhibitor has a concentration of about 0.1 μM to about 50 μM, preferably about 2 μM.

6. The method of claim 1, wherein the starting cell is contacted with the Tyk2 inhibitor and/or the TGFβ inhibitor for about 1 day to about 21 days or more.

7. The method of claim 1, further comprising providing at least one cardiomyocyte-inducing transcription factor and/or at least one cardiomyocyte-inducing microRNA to the starting cell.

8. The method of claim 7, wherein the at least one cardiomyocyte-inducing transcription factor is selected from the group consisting of MEF2C, TBX5, GATA4, MESP1, MYOCD, HAND2, SRF, ESRRG, ZFPM2, Nkx2.5, VEGF, Baf60c, and any combination thereof.

9. The method of claim 7, wherein the at least one cardiomyocyte-inducing transcription factor comprises MEF2C.

10. The method of claim 8, wherein the at least one cardiomyocyte-inducing transcription factor comprises TBX5.

11. The method of claim 8, wherein the at least one cardiomyocyte-inducing transcription factor comprises GATA4.

12. The method of claim 8, wherein the at least one cardiomyocyte-inducing transcription factor comprises MYOCD.

13. The method of claim 8, wherein the at least one cardiomyocyte-inducing transcription factor comprises MESP1.

14. The method of claim 7, wherein the at least one cardiomyocyte-inducing transcription factor comprises MEF2C, GATA4, MYOCD, and MESP1.

15. The method of claim 7, wherein the transcription factor and/or the microRNA is provided by an expression vector comprising a nucleotide sequence encoding at least one cardiomyocyte-inducing transcription factor and/or at least one cardiomyocyte-inducing microRNA, preferably, the expression vector is a viral vector, and more preferably, the viral vector is a lentiviral vector.

16. The method of claim 1, wherein the starting cell is a fibroblast, such as a skin fibroblast or a cardiac fibroblast.

17. A method for treating a cardiac disease in a subject, the method comprising administering to the subject at least one Tyk2 inhibitor and/or at least one TGFβ inhibitor.

18. The method of claim 17, wherein the cardiac disease is heart failure or myocardial infarction.

19. The method of claim 17, wherein the Tyk2 inhibitor is selected from the group consisting of Baricinib, Ruxolitinib, S-Ruxolitinib, Tofacitinib, Ocacitinib maleate, Itacitinib, Peficitinib, Gandotinib, FM-381, Filgotinib, PF-06826647, BMS-986165, or a structural analog thereof.

20. The method of claim 17, wherein the TGFβ inhibitor is selected from the group consisting of SB43152, TEW-7197, RepSox, GW788388, SD-208, LY364947, Y-27632, LDN-193189, LY2109761, and Galunisertib, or a structural analog thereof.

21. The method of claim 17, further comprising administering to the subject at least one cardiomyocyte-inducing transcription factor and/or at least one cardiomyocyte-inducing microRNA.

22. The method of claim 21, wherein the at least one cardiomyocyte-inducing transcription factor is selected from the group consisting of MEF2C, TBX5, GATA4, MESP1, MYOCD, HAND2, SRF, ESRRG, ZFPM2, Nkx2.5, VEGF, Baf60c, and any combination thereof.

23. The method of claim 21, wherein the at least one cardiomyocyte-inducing transcription factor comprises MEF2C.

24. The method of claim 22, wherein the at least one cardiomyocyte-inducing transcription factor comprises TBX5.

25. The method of claim 22, wherein the at least one cardiomyocyte-inducing transcription factor comprises GATA4.

26. The method of claim 22, wherein the at least one cardiomyocyte-inducing transcription factor comprises MYOCD.

27. The method of claim 22, wherein the at least one cardiomyocyte-inducing transcription factor comprises MESP1.

28. The method of claim 21, wherein the at least one cardiomyocyte-inducing transcription factor comprises MEF2C, GATA4, MYOCD, and MESP1.

29. The method of claim 21, wherein an expression vector comprising a nucleotide sequence encoding at least one cardiomyocyte-inducing transcription factor and/or at least one cardiomyocyte-inducing microRNA is administered, preferably, the expression vector is a viral vector, and more preferably, the viral vector is a lentiviral vector.

30. The method of claim 17, wherein the administration is topical, such as intracardiac.

31. The method of claim 17, wherein the administration is systemic.
